(19) United States
(12) Patent Application Publication
(10) Pub. No.: US 2018/0284580 A1
(43) Pub. Date: Oct. 4, 2018

(54) INTRA-ORAL CAMERA

(71) Applicant: Andrew Ryan Matthews, Redding, CA (US)

(72) Inventor: Andrew Ryan Matthews, Redding, CA (US)

(21) Appl. No.: 15/945,458

(22) Filed: Apr. 4, 2018

Related U.S. Application Data
(63) Continuation-in-part of application No. 15/471,632, filed on Mar. 28, 2017, now Pat. No. 9,939,714.

(51) Int. Cl. G03B 17/56; G02B 23/08; A61B 1/24; A61B 1/00; A61B 1/05; H04M 1/02

(52) U.S. Cl. CPC G03B 17/565; G02B 23/08; H04M 1/0264; A61B 1/00197; A61B 1/05; A61B 1/24

(57) ABSTRACT

A camera attachment device includes a mounting ring for attachment over a camera lens of a mobile phone. A periscope module includes a first periscope end for attachment to the mobile phone at the mounting ring. A second periscope end is for capturing images. A periscope tube is connected between the first periscope end and the second periscope end. A light collector is attached to the mounting ring. The light collector is shaped to extend over a light source from the mobile phone. The light collector is configured to receive light from the light source and direct the light through the mounting ring and into the first periscope end. The light exits the second periscope end so as to provide illumination for capturing the images.

### BACKGROUND

Cameras on mobile phones are able to capture photographs and videos. Most cameras on mobile phones have fixed-focus lenses and sensors whose small size limit their performance in poor lighting. Photoflash is often provided by a light-emitting diode (LED) source which illuminates less intensely over a much longer exposure time than a traditional flash strobe.

### BRIEF DESCRIPTION OF THE DRAWINGS

FIG. 1 shows a mobile phone with a camera and LED flash source prepared for mounting a periscope module in accordance with an embodiment.

FIG. 2 shows a periscope module mounted on a mobile phone in accordance with an embodiment.

FIG. 3 shows a light tube connected to a periscope module mounted on a mobile phone in accordance with an embodiment.

FIG. 4 shows a light tube connected to a periscope module ready to be mounted on a mobile phone in accordance with an embodiment.

FIG. 5 shows a periscope module mounted on a mobile phone used as an intra-oral camera in accordance with an embodiment.

FIG. 6, FIG. 7, FIG. 8 and FIG. 9 show various configurations of a periscope that can be used with an intra-oral camera.

FIG. 10 shows a disassembled view of a periscope in accordance with an embodiment.

FIG. 11 shows an assembled view of the periscope shown in FIG. 9 in accordance with an embodiment.

### DESCRIPTION OF THE EMBODIMENT

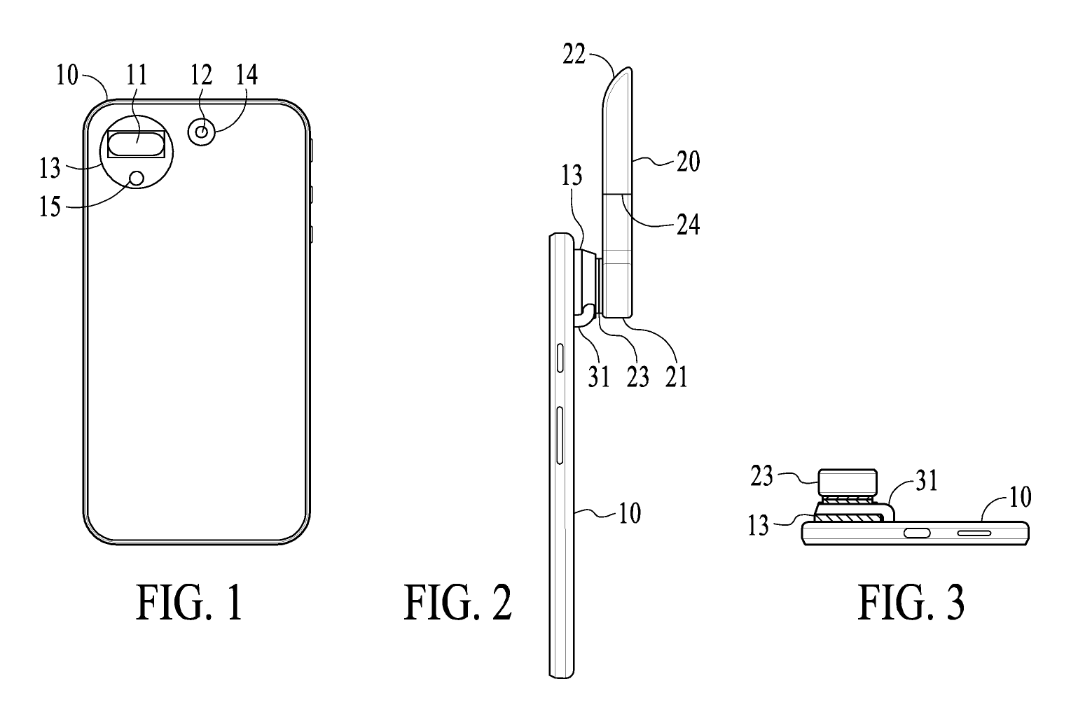

FIG. 1 shows a mobile phone 10. Around a camera lens 11 is placed a periscope mounting ring 13 with a magnet 15 used to hold a periscope module in place. A ring attachment 14 located around LED light source 12 has a phone adherence portion that adheres to mobile phone 10 and a light tube adherence portion that adheres to a light tube 31, shown in FIG. 3. The light tube adherence portion rotates with respect to the phone adherence portion so that light tube 31 when attached to the light tube adherence portion can rotate with respect to LED light source 12.

FIG. 2 shows a periscope module 20 mounted on mobile phone 10 at periscope mounting ring 13. Magnet 15 holds periscope module 20 to periscope mounting ring 13, making it simple to attach and detach periscope module 20 to mobile phone 10. A swivel ring 23 allows periscope module 20 to rotate 360 degrees with respect to mounting ring 13. This allows positioning of periscope module 20 when mobile phone 10 and periscope module are used as part of an intra-oral camera. Periscope module 20 includes a periscope reflector 21 and a periscope reflector 22 that directs light within periscope module 20. A swivel point 24 allows swiveling of the periscope 20 to allow adjustment of positioning of periscope reflector 22. This ability to swivel 360 degrees allows a user to rotate to a 90-degree view for a Buccal image, or 180 Degrees for an upper or Maxilla image when using camera 10 and periscope module 20 as an intra-oral camera.

FIG. 3 shows a top view of mobile phone 10 with swivel ring 23 connected above periscope mounting ring 13. A light tube 31 directs light from LED light source 12 of mobile phone 10 into periscope module 20. This provides illumination that allows mobile phone 10 and periscope module 20 to together act as an intra-oral camera. Light tube 31 is held against LED light source 12 by ring attachment 14.

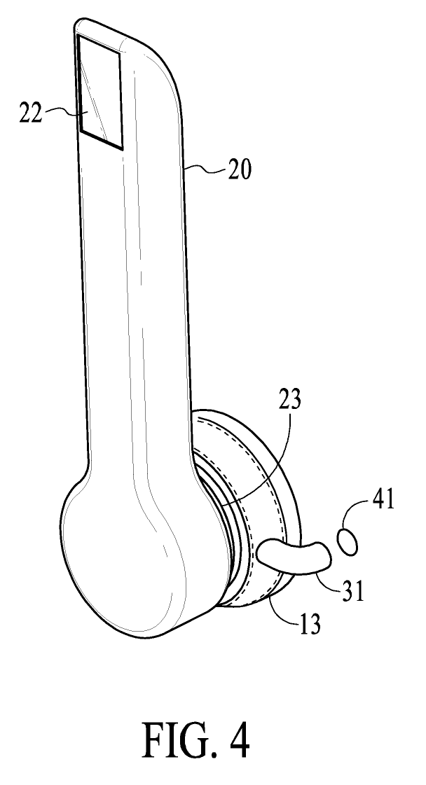

FIG. 4 provides another view of periscope module 20 and light tube 31. Light tube 31 enters periscope module 20 between periscope mounting ring 13 and swivel ring 23. Swivel ring 23 is connected above periscope mounting ring 13. Light from LED light source 12 exits periscope module 20 from periscope reflector 22. Light tube 31 is able to rotate around swivel ring 23 to be positioned to accommodate various locations of a LED light source on different types of mobile phones. For example, a clicked swivel embodiment allows locking light tube 31 into position to accommodate a location of a LED light source on a mobile phone. This allows periscope module 20 to be adapted to use on a variety of models of mobile phones available from various manufacturers. A diameter of light tube 31 is selected to be greater than or equal to a diameter of an opening 41 of the light source providing light to light tube 31.

For example, periscope module 20 is constructed by a tube portion 51 located between a 90-degree turning periscope mobile phone camera lens 52 and a 90-degree turning periscope mobile phone camera lens 53. For example, one or both of 90-degree turning periscope mobile phone camera lens 52 and 90-degree turning periscope mobile phone camera lens 53 are implemented from a periscope mobile phone camera lens model A-8022 available from Seaflower Technology Company Limited, in Guangdong, China. For example, periscope mobile phone camera lens 53 is able to swivel 360 degrees with respect to tube portion 51 allowing a user to rotate to a 90-degree view for a Buccal image, or 180 Degrees for an upper or Maxilla image when using camera 10 and periscope module 20 as an intra-oral camera. Rotation is, for example, alternative to, or in addition to swivel point 24. For example, periscope module 20 is made of materials that can be sterilized in an autoclave at or above 132° C. (270° F.) allowing for sterilization when during use periscope module 20 becomes contaminated. In order to allow this, periscope module 20 and other parts used to form the mobile intra-oral camera are made with durable resins and metals that can withstand the heat used for sterilization and that can withstand chemical disinfection surface wipes. For example, a plastic barrier sleeve can be used to cover all or part of periscope module 20 during use to prevent contamination.

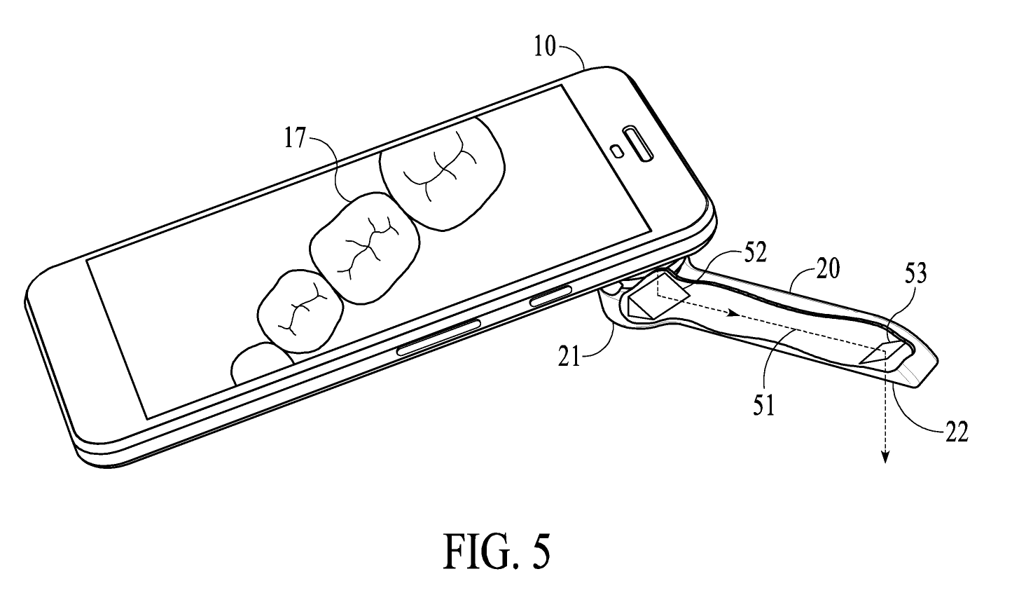

FIG. 5 illustrates mobile phone 10 and periscope module 20 activating together as an intra-oral camera. Light from LED light source 12 travels through periscope module 20 and provides illumination to teeth in a patient's mouth. A visual image of the teeth returns back through periscope module 20 and is captures by camera lens 11 of mobile phone 10. An image of the teeth is shown on a screen 17 of mobile phone 10. Pictures or video of the teeth can be recorded by mobile phone 10. For example, an intra-oral camera app is response to voice commands such as “snap picture”, “start video”, “stop video”, “increase light”, “decrease light” and so on.

For the purposes of illustration, periscope module 20 has been shown to have a fairly straightforward configuration. However, depending on application requirements, periscope module 20 can use any configuration convenient for implementing an intra-oral camera.

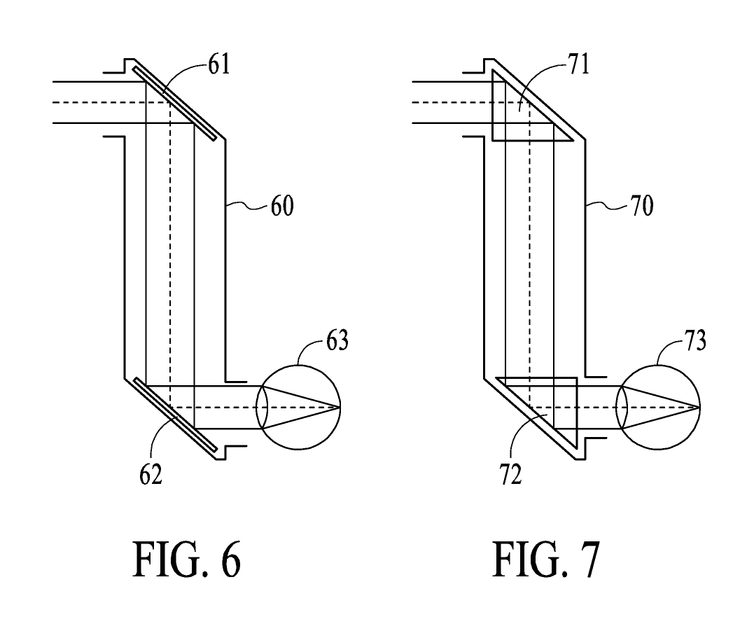

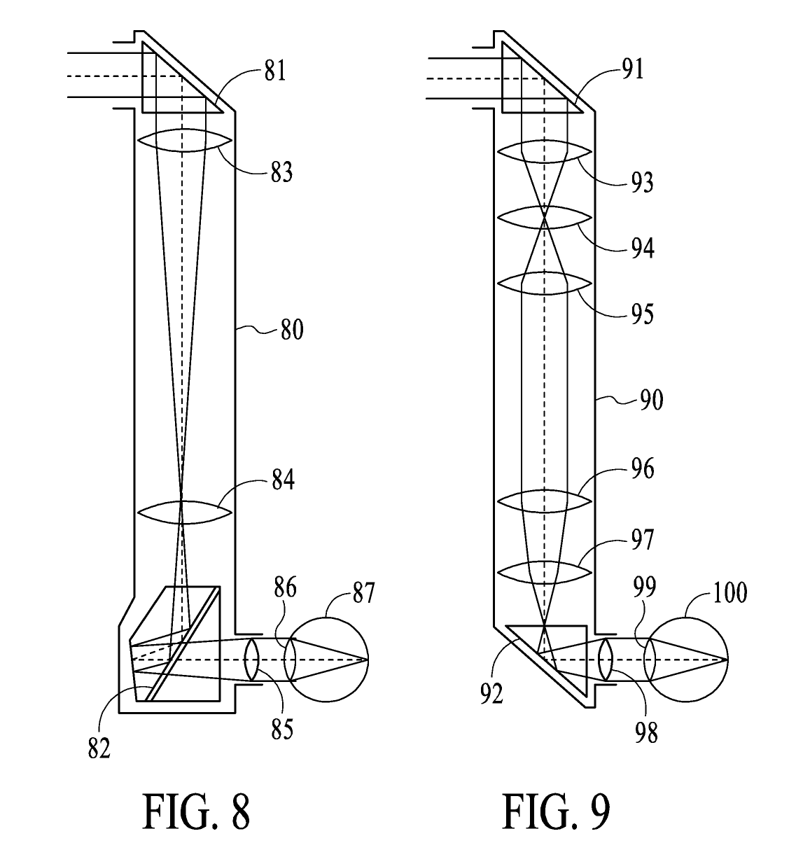

For example, FIG. 6 shows a periscope 60 implemented using a mirror reflector 61 and a mirror reflector 62 to focus an image at a camera lens location 63.

For example, FIG. 7 shows a periscope 70 implemented using a prism 71 and a prism 72 to focus an image at a camera lens location 73. For example, FIG. 8 shows a periscope 80 implemented using a prism 81, a prism 82, a lens 83, a lens 84, a lens 85, and a lens 86 to focus an image at a camera lens location 87. For example, FIG. 9 shows a periscope 90 implemented using a prism 91, a prism 92, a lens 93, a lens 94, a lens 95, a lens 96, a lens 97, a lens 98 and a lens 99 to focus an image at a camera lens location 100. Within the lenses in any of the implementations, magnification can be added, as desired, to obtain a desired image size.

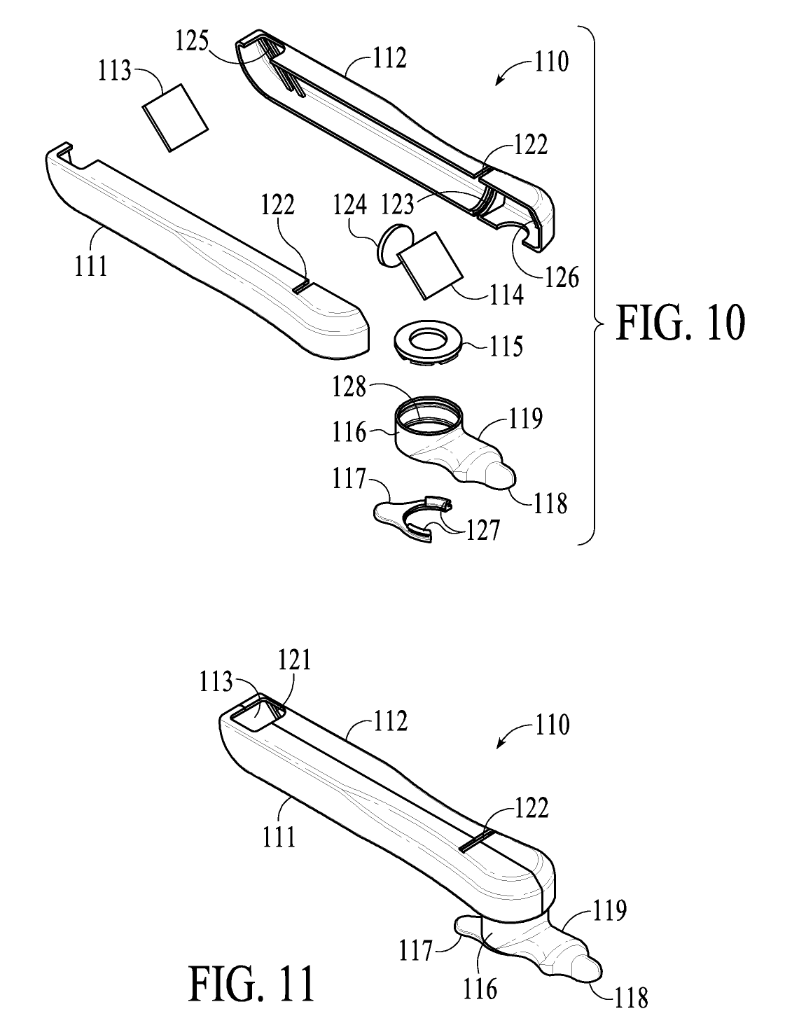

Parts of a periscope 110 are shown disassembled in FIG. 10 and assembled in FIG. 11. When assembled, casing portion 111 is joined to casing portion 112. A mirror 113 is held in place by a slot 125 of casing portion 112 and a similar slot of casing portion 111. A mirror 113 is held in place by a slot 126 of casing portion 112 and a similar slot of casing portion 111.

A lens 124 is held in place by a slot 123 of casing portion 112 and a similar slot of casing portion 111. When assembled casing portion 111 and casing portion 112 form a periscope module case. Lens 124 may be removed or replaced after casing portion 111 is joined to casing portion 112 through a slot 122 formed by both casing portion 111 and casing portion 112, as is shown in FIG. 11. The ability to replace lens 124 allows for changes in, for example, magnification and focal length depending on requirements of a particular application.

When assembled, inner wheel part 115 is attached to mounting ring 116 providing rotatable connection of mounting ring 116 to the assembled periscope module case. For example, when attached to a smart phone, mounting ring 116 is placed over the camera lens of the smart phone. A light collector end 118 of a light collector 119 is placed over the LED light source of the smart phone camera. The light from the LED light source travels through light collector 119 through mounting ring 116, through the inside of the assembled periscope module case and is reflected off mirror 113 to provide illumination for capturing images configured as pictures or video. For example, light collector 119 includes a light tube within light tube housing. The light tube directs light through mounting ring 116 to mirror 114. A tab 117 can be attached to the smartphone. The attachment can be, for example, through a magnetic bond, through use of an adhesive, or through a mechanical mechanism. Helical threading 128 of mounting ring 116 and outer helical threading 127 of tab 117 are used to attach mounting ring 116 to tab 117 so that mounting ring 116 is placed over the camera lens of the smart phone and light tube casing end 118 is placed over the LED light source of the smart phone camera. The assembled periscope module case can rotate around mounting ring 116 to a desired angle for capturing images.

The foregoing discussion discloses and describes merely exemplary methods and embodiments. As will be understood by those familiar with the art, the disclosed subject matter may be embodied in other specific forms without departing from the spirit or characteristics thereof. Accordingly, the present disclosure is intended to be illustrative, but not limiting, of the scope of the invention, which is set forth in the following claims.

What is claimed is:

1. A periscope attachment for an intra-oral camera, comprising:
a mounting ring for attachment over a camera lens of a mobile phone;
a periscope module including:
a first periscope end for attachment to the mobile phone at the mounting ring,
a second periscope end for capturing intra-oral images, and
a periscope tube connected between the first periscope end and the second periscope end; and
a light collector attached to the mounting ring, the light collector being shaped to extend over a light source from the mobile phone, the light collector being configured to receive light from the light source and direct the light through the mounting ring and into the first periscope end, the light exiting the second periscope end so as to provide illumination for capturing the intra-oral images;
wherein the periscope module is attached to the mounting ring so that the periscope tube is rotatable with respect to the light tube cavity to allow configuration of a desired angle of the periscope tube for capturing the intra-oral images.

2. A periscope attachment as in claim 1, wherein the mounting ring allows swiveling of the periscope tube of 360 degrees.

3. A periscope attachment as in claim 1, wherein the light collector comprises a light tube.

4. A periscope attachment as in claim 1, wherein the light collector comprises a light tube within light tube housing.

5. A periscope attachment as in claim 1, wherein the first periscope end includes a click dial for locking in a rotation angle.

6. A periscope attachment as in claim 1, wherein the periscope tube includes a swivel point that allows 360 swiveling.

7. A periscope attachment as in claim 1, wherein material used to construct the periscope attachment include durable resins and metals that can withstand heat at or above at or above 132° C. (270° F.) used for sterilization and that can withstand chemical disinfection surface wipes.

8. An intra-oral camera, comprising:
a mobile phone including a camera lens and a light source;
a mounting ring for attachment over the camera lens;
a periscope module including:
a first periscope end for attachment to the mobile phone at the mounting ring,
a second periscope end for capturing intra-oral images, and
a periscope tube connected between the first periscope end and the second periscope end; and
a light collector attached to the mounting ring, the light collector being shaped to extend over the light source, the light collector being configured to receive light from the light source and direct the light through the mounting ring and into the first periscope end, the light exiting the second periscope end so as to provide illumination for capturing the intra-oral images;
wherein the periscope module is attached to the mounting ring so that the periscope tube is rotatable with respect to the light tube cavity to allow configuration of a desired angle of the periscope tube for capturing the intra-oral images.

9. An intra-oral camera as in claim 8, wherein the light collector comprises a light tube.

10. A periscope attachment as in claim 8, wherein the light collector comprises a light tube within light tube housing.

11. A intra-oral camera as in claim 10 wherein a click dial is used for locking in a rotation angle of the periscope tube.

12. An intra-oral camera as in claim 8 wherein the periscope tube includes a swivel point that allows 360 swiveling.

13. A intra-oral camera as in claim 8 wherein the mounting ring allows swiveling 360 degrees.

14. A camera attachment device, comprising:
a mounting ring for attachment over a camera lens of a mobile phone;
a periscope module including:
a first periscope end for attachment to the mobile phone at the mounting ring,
a second periscope end for capturing images, and
a periscope tube connected between the first periscope end and the second periscope end; and
a light collector attached to the mounting ring, the light collector being shaped to extend over a light source from the mobile phone, the light collector being configured to receive light from the light source and direct the light through the mounting ring and into the first periscope end, the light exiting the second periscope end so as to provide illumination for capturing the images.

15. A periscope attachment as in claim 14, wherein the light collector comprises a light tube.

16. A periscope attachment as in claim 14, wherein the light collector comprises a light tube within light tube housing.

17. A camera attachment device as in claim 14, wherein the periscope tube includes a swivel point that allows 360 swiveling.

18. A camera attachment device as in claim 14, wherein the periscope module is attached to the mounting ring so that the periscope tube is rotatable with respect to the light tube cavity to allow configuration of a desired angle of the periscope tube for capturing the images.

19. A camera attachment device as in claim 18 wherein the first periscope includes a click dial connected to the light tube for locking in a rotation of a light tube at a specific location.

20. A camera attachment device as in claim 14, wherein material used to construct the camera attachment device include durable resins and metals that can withstand heat at or above at or above 132° C. (270° F.) used for sterilization and that can withstand chemical disinfection surface wipes.
